(19) United States
(12) Patent Application Publication
(10) Pub. No.: US 2023/0390064 A1
(43) Pub. Date: Dec. 7, 2023

(54) PENILE IMPLANT WITH EXTERNALLY CONTROLLED PIEZOELECTRIC PUMP

(71) Applicant: Igor Pashin, Wheeling, IL (US)

(72) Inventors: Mikhail A. Alexsandrov, Saint-Petersburg (RU); Nikolai V. Smirnov, Saint-Petersburg (RU); Maria A. Dontsova, Saint-Petersburg (RU)

(21) Appl. No.: 17/834,040

(22) Filed: Jun. 7, 2022

(51) Int. Cl. A61F 2/26

(52) U.S. Cl. CPC A61F 2/26; A61F 2250/0003

(57) ABSTRACT

A medical human implant is proposed. The device is intended as an improvement over the existing implant systems used to treat erectile dysfunction, replacing a hand-operated pump. The device includes two piezoelectric pumps or a single piezoelectric pump with a reverse mode, an implant controller, and a receiver antenna of a wireless charger. The remotely operated implant controller is electrically coupled to the pump and is configured to actuate the pump to circulate fluid within the modifiable implant system. The wireless charger receiver provides powering of both the pump and controller, wherein energy is transferred from an external, non-implantable, source.

### BACKGROUND OF THE INVENTION

### Field of the Invention

The present invention is related to penile implants, and, more particularly, to implants that use an externally controlled piezoelectric pump.

### Description of the Related Art

The present invention is intended to allow men with erectile dysfunction choose the most suitable driver for an inflatable penile implant. Conventional penile implants are implemented as a male organ prosthesis comprising two inflatable cylinders (2) implanted into both corpus cavernosa of the penis, a fluid-filled tank (1) implanted into the abdominal cavity and connected to the cylinders, and a hand-operated pump (3) often placed inside the scrotum and used for transferring fluid from the tank (1) into the cylinders (2) thus inflating them. Exemplary penile implants of this kind include AMS 700 and Inflatable medical implant system (U.S. Pat. No. 10,660,757). FIG. 1 shows a conventional implant system, such as ones sold under the brand name AMS 700.

Conventionally, an AMS 700 user has to squeeze the bulb of the pump (3) repeatedly to draw fluid from the tank (1) (sometimes called reservoir or volume) and transfer it to cylinders (2), creating an erection. The user may then make the penis flaccid once again by selectively releasing the fluid back into the tank.

However, some users may face difficulties when using the hand-operated pump due to their physiological limitations, such as complete or partial absence of upper limbs, decreased finger mobility or impaired dexterity, possibly, because of arthritis in the hand or other conditions causing loss of fine motor skills. Such users may find it difficult to handle the bulb of the pump or squeeze it repeatedly.

Penile implants are proven to effectively alleviate erectile dysfunction in men, however, users and surgeons would welcome further improvements of these devices.

Thus, a modification for existing systems is proposed, wherein the hand-operated pump would be replaced with an automatic one.

### SUMMARY OF THE INVENTION

Accordingly, a penile implant with a piezoelectric pump is proposed to address he disadvantages of the related art.

Additional features and advantages of the invention will be set forth in the description that follows, and in part will be apparent from the description, or may be learned by practice of the invention. The advantages of the invention will be realized and attained by the structure particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF DRAWINGS

The accompanying drawings, which are included to provide a further understanding of the invention and are incorporated in and constitute a part of this specification, illustrate embodiments of the invention and, together with the description, serve to explain the principles of the invention.

In the drawings:

FIG. 1 shows a conventional implant system.

FIG. 2 is a cross-sectional diagram of the automatic cylinder-pumping module.

FIG. 3 is a diagram of the automatic cylinder-pumping module

FIG. 4 is a diagram of mechanical connections of the automatic pump module.

FIG. 5 illustrates activation of the penile implant with an automatic pump.

FIGS. 6-8 show various arrangements of the pump(s).

FIG. 9 shows an exemplary embodiment with two unidirectional piezoelectric pumps mounted in a single case, wherein each pump works in a single fluid transfer direction.

FIG. 10 shows an exemplary remote controller for the pump.

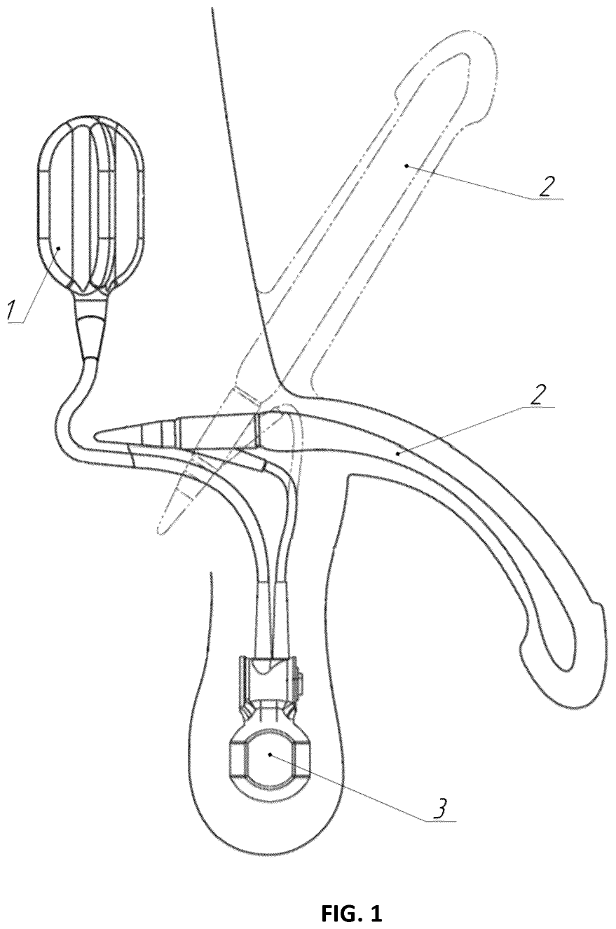

### DETAILED DESCRIPTION OF EMBODIMENTS OF THE INVENTION

Reference will now be made in detail to the preferred embodiments of the present invention, examples of which are illustrated in the accompanying drawings.

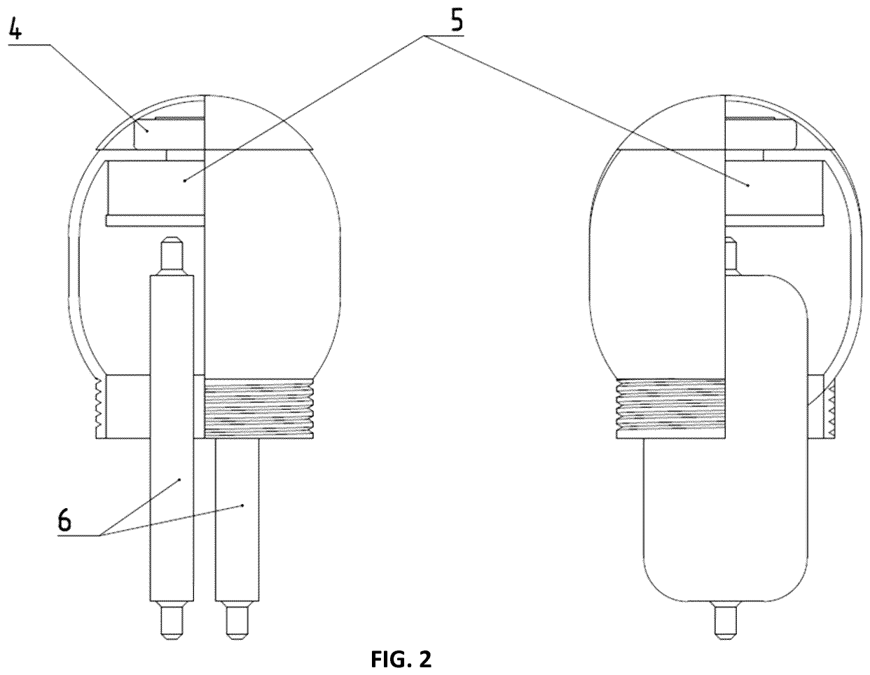

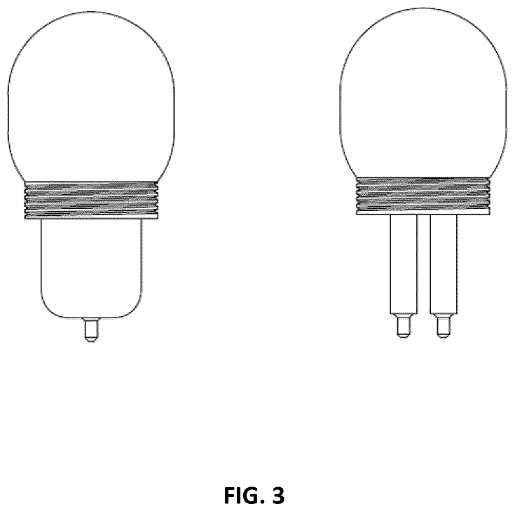

The present invention can be implemented as a ready-made upgraded implant or as an auxiliary module for the already implanted penile prosthesis. FIG. 2 is a diagram of the automatic cylinder-pumping module.

The device is designed to easily replace the hand-operated pump in existing penile prostheses, so that any man could use it regardless of their physical limitations. The dimensions of the proposed device are typically limited to about 50 mm in height, and about 25 mm in width and length. The device meets medical and implant safety requirements.

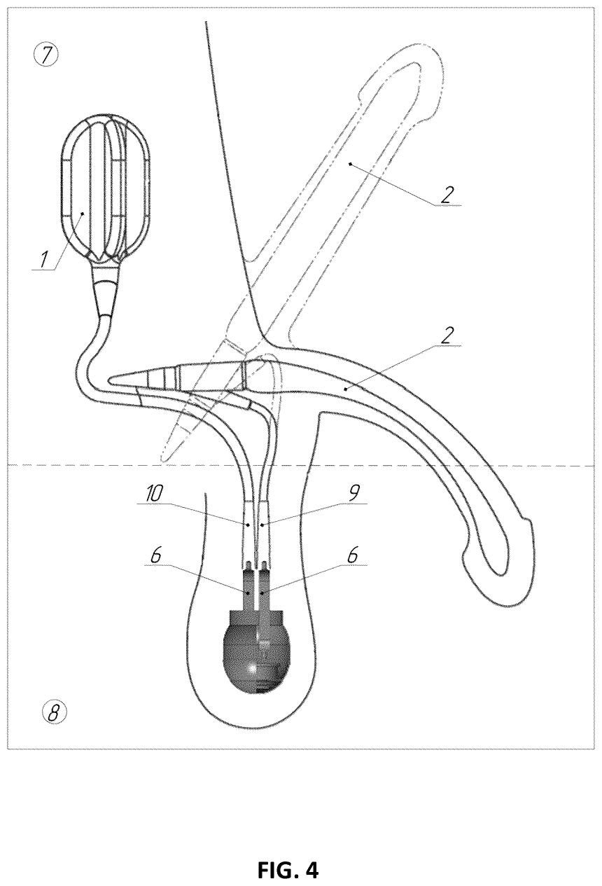

The automatic pump module is surgically mounted into the already implanted penile prosthesis in the area, where the hand-operated pump (8) is located, without affecting other components of the prosthesis (7). FIG. 4 is a diagram of mechanical connections of the automatic pump module. After the hand-operated pump is removed, the automatic pump module is mounted so as to have sealed fluid coupling with the tank (1) via a tube (10), and with the cylinders (2) via a tube (9).

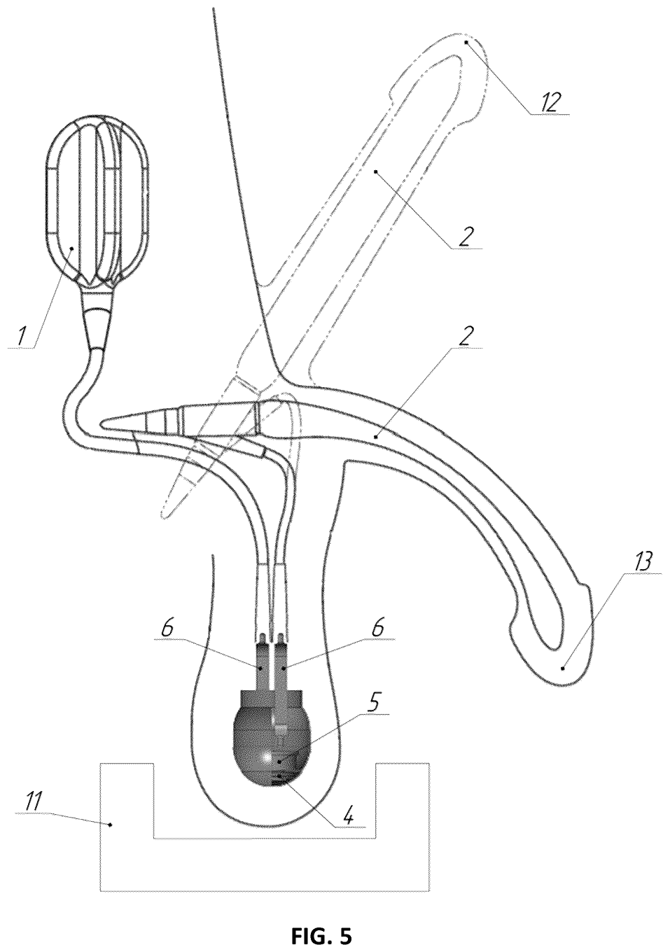

After the pump has been replaced, the penile implant system can be fully used again. The system can operate in three states: cylinder pumping (creating an erection), stand-by (no fluid circulation in the system), and tank pumping (making the penis flaccid). FIG. 5 is an operation diagram for a penile implant with an automatic pump module.

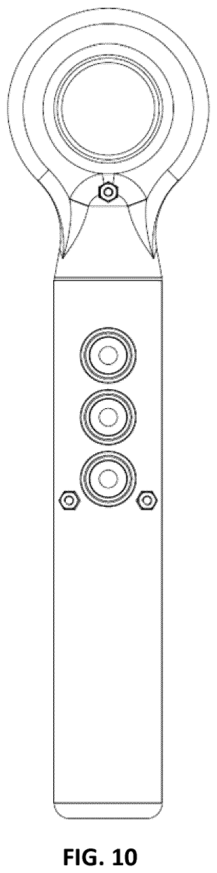

In the cylinder pumping state, the system perform the following steps: First, the user has to activate the external power source acting as a remote control (11) by moving the remote control as close as possible to the area where the automatic pump module is located. FIG. 10 shows an exemplary remote controller for the pump.

The remote control is equipped with a directed wireless charger antenna. Power is transferred from the remote control to the receiver (4) in the automatic pump module, whereby a controller (5) is activated, which, in turn, actuates the piezoelectric pump or pumps (6). The pump (6) transfers fluid from the tank (1) via connective tubes and directly though the automatic pump module into the cylinders (2). The pumps (6) are piezoelectric bi-directional fluid pumps capable of transferring liquid in either direction until the maximum pressure in the system is reached, thus preventing the user from intentionally breaking the implant system or getting physically injured.

During the fluid transfer, the penile implant changes its state from flaccid (13) into erect (12). The pressure level in the cylinders (2) is regulated by the user (via powering time of the automatic pump), or it rises until the maximum pressure is reached. After the fluid transfer is finished, the automatic pump module does not need power anymore, so the remote control (11) can be put away.

In the stand-by state, the system is not powered by an external power source, i.e. the automatic pump module is turned off. The penile implant itself can be either flaccid (13) or erect (12).

In the tank pumping state, the system performs the same steps as in the cylinder pumping state, but in reverse. The fluid is transferred from the cylinders (2) via the tubes and directly through the automatic pump module back into the tank (reservoir) (1). As the fluid leaves the cylinders, the penile implant goes from an erect state (12) into a flaccid state (13). (Note that the figures show the element (2) as a single component, although in many practical systems there are two such cylinders there. As an alternative, a single such cylinder may be used.)

The pumping direction is regulated by a microcontroller on the driver's board. The microcontroller receives impulses of certain frequency transmitted over the power circuit. The incoming impulses briefly, for a fraction of msec, stop power coming to the pump driver, but do not crash the system, since the driver comprises an additional electrolytic capacitor maintaining stable operation of the automatic pump. Since the pumps and driver consume just a few mA, a capacitor of minimum size, much smaller than SMD standard, can be installed in order to conform to limited space. The signal to reverse pumping direction is generated by pressing the corresponding button on the remote control panel of the external power source.

The main improvement of the proposed invention is how the elements of the automatic system are arranged. In prior art, penile prostheses with automatic pumping systems mainly comprise a pump, a controller, a fluid pressure or pump voltage sensor that controls fluid transfers. The whole system is battery-powered. All components have to be implanted together, and no possible way of modifying or servicing the system is provided, which are clearly drawbacks of the prior art.

The proposed automatic pump does not have components that could quickly wear or fail, such as fluid pressure or pump voltage sensors and batteries, which dramatically increases the service life of the system and its safety. It is widely known that batteries are prone to quick wear and premature discharging, and there are currently no batteries that do not require replacement (which in the given case means surgery), are reliable enough, and do not pose threat to the user in case of their sudden failure due to external factors, such as a physical impact. Also, without an implanted battery, the automatic device would be smaller.

Instead of an implanted battery, it is proposed to install a small-sized non-degradable receiver that provides unlimited work cycles. This, the automatic device is powered by an external, non-implantable, battery (a remote control with a charger antenna) that can be easily modified or replaced when needed.

Another component that has been removed in the proposed solution is the fluid pressure or pump voltage sensor. Instead, the features of the pump are employed. The pump of the proposed automatic system is a piezoelectric pump without complex kinematics or easily-worn components. Since the piezoelectric pump is selected to accommodate a pre-determined output fluid pressure that would create an erection safely for the user, it does not require any additional sensors. As soon as a pre-determined pressure in cylinders is reached, the piezoelectric pump won't be able to transfer liquid anymore and thus increase the pressure further, while the remaining fluid from the tank will continue to circulate inside the pump. Therefore, the system will not fail or be subjected to an extreme pressure leading to faster wearing, which is the case with any other pump type (mechanical pumps in particular). Mechanical pumps are unadvisable, too, even though their sizes can be small, because they comprise rotating elements and contacts that grate against each other. In addition, such pumps cannot sustain dynamic loads that are unavoidable in a person's daily life.

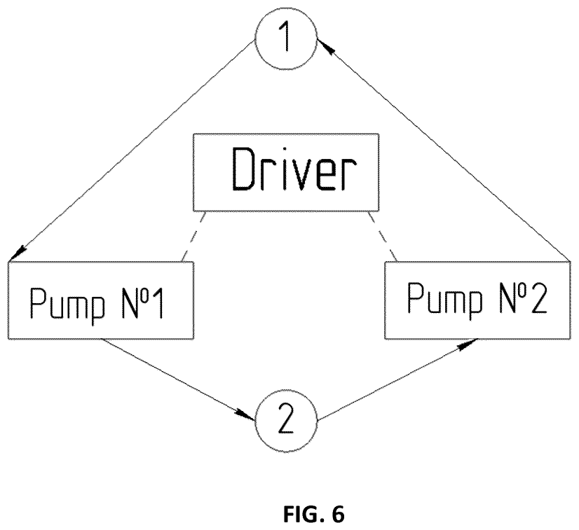

A piezoelectric pump can be installed into the system in different ways. One way (see FIG. 6) involves using two unidirectional piezoelectric pumps mounted in a single case, wherein each pump works in a single fluid transfer direction, i.e. “tank→pump→cylinders” or “cylinders→pump→tank”, respectively. Such scheme allows to use a simple pump which is easier to produce and simple to use. The resulting system is fully automatic. The case can be made of titanium, which solves the paramount biocompatibility issue.

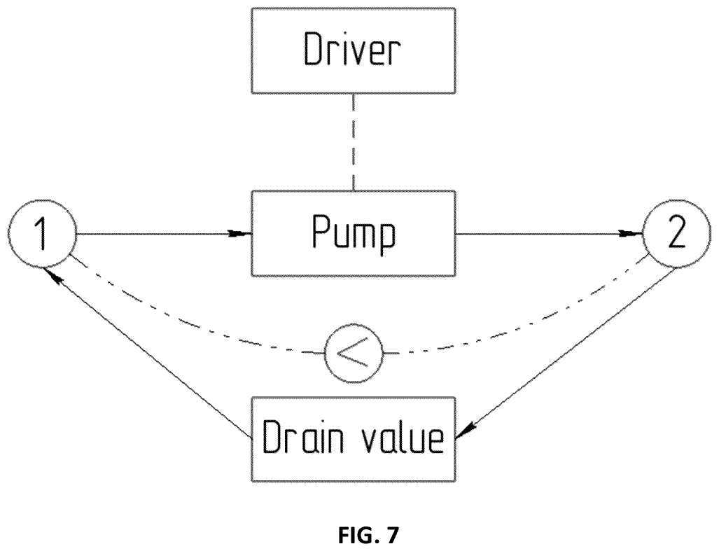

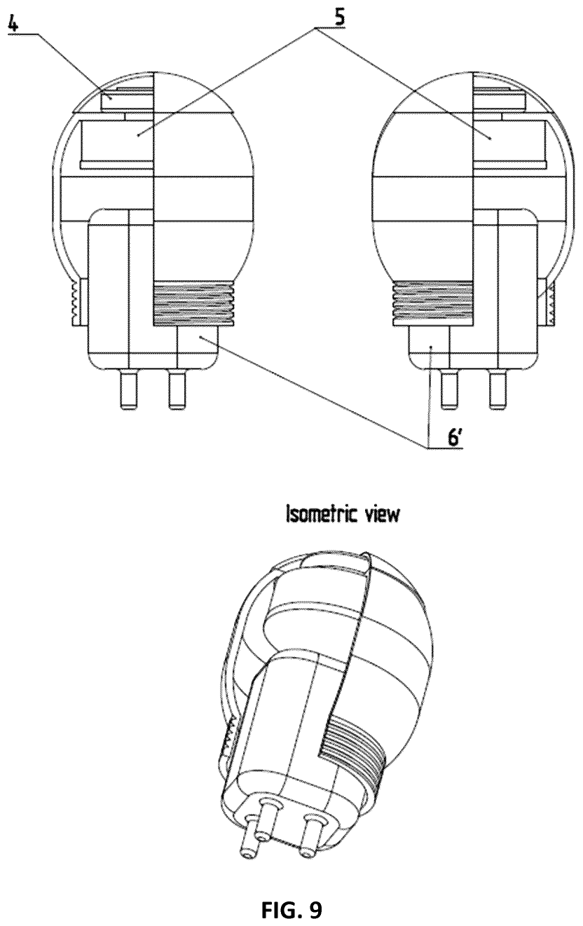

Another installation configuration includes mounting a piezoelectric pump into the implant system (see FIGS. 7 and 9) by using a single unidirectional piezoelectric pump 6′ with an integrated drain valve. Such system would be semi-automatic, as a similar mechanic valve is used in conventional hand-operated pumps. It operates as follows: the piezoelectric pump automatically transfers fluid from the tank into the cylinders, while in order to transfer it back into the tank, the user has to open the drain valve. When opened, it lets the fluid to naturally flow from the cylinders into the tank because of pressure difference. Automatic valves (e.g. solenoid valves) can also be used. In this case, the system would require no manual manipulations at all; however, additional mechanical parts may affect reliability of the system.

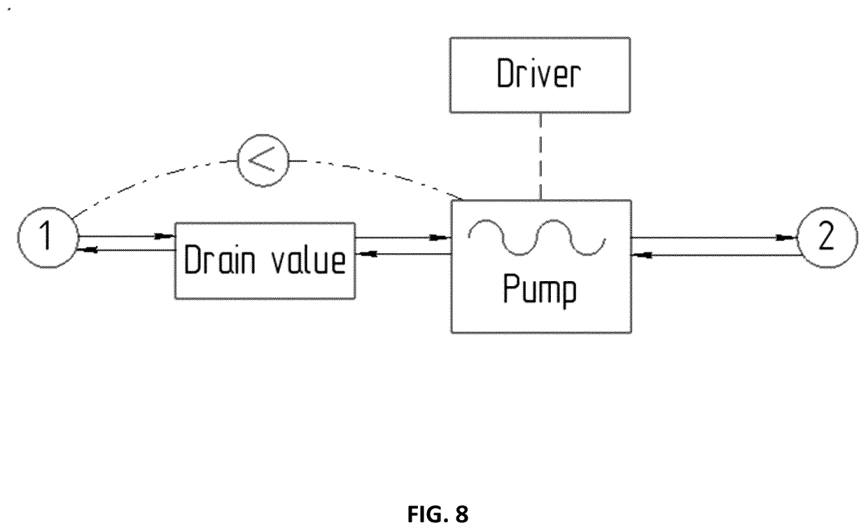

Yet another way of installing a piezoelectric pump into the implant system (see FIG. 8) involves using a single bi-directional pump with a sine-like piezoelectric element. Phase currents in such pump have sinusoidal shapes. To increase intensity, an autonomous voltage inverter with pulse width modulation is used. It operates as follows: the sinusoidal piezoelectric element can change its amplitude and phase depending on the voltage, and so it can “behave” like a wave, moving in either direction and thus making the fluid flow in the same direction. The resulting system is fully automatic. The piezoelectric pump operates as follows: after a controller signal has been received, that determines the fluid transfer direction, the fluid starts to circulate, depending on the mode, either in the “tank→pump→cylinders” direction or in “cylinders→pump→tank” direction. The main advantage of this piezoelectric pump is that it is small and requires little power.

As the embodiment using a single bi-directional piezoelectric pump is meant to be fundamental for the proposed invention and the present disclosure, the system control method is described based on this embodiment.

FIG. 10 shows an exemplary remote controller (11) for the pump. The controller (11) includes a transmitter antenna (also typically in the form of a toroid coil), a power supply controller, three control buttons, a battery, a voltage transformer to convert 5 V DC to 12 V DC needed for the pump.

The buttons on the remote controller perform the functions of: 1— direct (forward) pumping, 2— reverse pumping, 3— stop (no pumping in either direction). The stop function permits “remembering” the last state of the system, so that when the power is again provided, there is no unintentional pumping by the piezoelectric pump. The pump controller that is implanted also permits such a configuration, storing the information.

The battery in the remote controller is a rechargeable battery that is easily replaceable by the user, unlike implanted batteries, which normally cannot be replaced without surgery.

Since the invention is used in the field of implantable medical devices, the materials that are used need to be mechanically strong, corrosion-resistant, biochemically inert, non-allergenic, non-carcinogenic and non-mutagenic. The piezopump needs to be highly mechanically stable, since this mechanical device is subject to wear and friction. Titanium of a high purity (e.g., 99,2% Ti or 99,5% Ti) is preferred. The connecting tubes, reservoirs and cylinders of the system are preferably made from elastic materials, which have a long useful life and are biocompatible. The elasticity is needed because the various tubular members need to bend easily during use, due to the physiological specifics of the human body, organ structure and movement of the implantee. They also should not cause any discomfort for the implantee. The reservoir (1) and the cylinders (2) need to be flexible/elastic as well, to enable multiple fill/drain cycles. As one example, silicone elastomer that includes multiple layers of polydimethylsiloxane can be used.

Additionally, the entire module with the pump, the controller, the reservoir and all the tubular members can be enclosed in a membrane (not shown in the figures). This material should be biocompatible, as well as transparent for the electromagnetic fields used to charge and operate the piezopump, the receiver and the controller. As an example, silicone elastomer can be used as an enclosure/membrane, since this material is simple to use and creation of complex shapes with it is straightforward. The enclosure should be hermetically sealed.

The remote controller (11) is expected to frequently come into contact with the implantee's skin, and, as such, should be made of hypoallergenic material. Here, as an example, PLA plastic may be used as the material.

Having thus described a preferred embodiment, it should be apparent to those skilled in the art that certain advantages of the described method and apparatus have been achieved. It should also be appreciated that various modifications, adaptations, and alternative embodiments thereof may be made within the scope and spirit of the present invention.

References (all incorporated herein by reference in their entirety):
https****www.bostonscientific.com/en-US/medical-specialties/urology/products/ams-700-inflatable-penile-prosthesis.html
https****genital-clinic.ru/protezirovanie-polovogo-chlena/
Inflatable medical implant system:
   https****pdfpiw.uspto.gov/.piw?PageNum=0&docid=10376366&IDKey=DE8ECA976840%0D%0A&HomeUrl=%2F%2Fpatft.uspto.gov%2Fnetahtml%2FPTO%2Fpatimg.htm
Method of implanting a penile prosthetic in treating erectile dysfunction:
   https****pdfpiw.uspto.gov/.piw?PageNum=0&docid=10660757&IDKey=3D40FE078C6E%0D%0A&HomeUrl=%2F%2Fpatft.uspto.gov%2Fnetahtml%2FPTO%2patimg.htm

What is claimed is:

1. A penile implant, comprising:
first and second cylinders fillable with liquid from a reservoir;
the first and second cylinders connected to the reservoir through first and second connecting channels;
a piezoelectric pump between the reservoir and the first and second cylinders, having the first connecting channel as an input from the reservoir, and the second connecting channel as an output to the first and second cylinders, such that the output splits to fill first and second cylinders;
the piezoelectric pump including a receiver and a controller;
the receiver configured to be activated when placed in an electromagnetic field; and
the controller receiving energy from the receiver when the receiver is activated, and the controller activating the piezoelectric pump to either inflate the first and second cylinders with liquid from the reservoir, or to deflate the first and second cylinders by draining the liquid from the first and second cylinders into the reservoir.

2. The implant of claim 1, wherein the piezoelectric pump includes a first piezoelectric pump and a second piezoelectric pump, wherein the first piezoelectric pump is used to fill the first and second cylinders with the liquid, and the second piezoelectric pump is used to drain the first and second cylinders.

3. The implant of claim 1, wherein the receiver is located on an opposite side of the input and the output, and wherein the controller is located between the receiver and the input and the output.

4. The implant of claim 1, wherein the receiver is shaped as an induction coil shaped as a toroid.

5. The implant of claim 1, wherein the receiver, when activated, is coupled to a remote controller with a toroid-shaped induction coil antenna.

6. The implant of claim 1, wherein the piezoelectric pump is configured to pump up to 100 to 120 ml/min.

7. The implant of claim 1, wherein the piezoelectric pump is configured to pump when supplied with −50 volt DC to +200 volt DC.

8. The implant of claim 1, wherein the implant consumes 400-450 milliwatt at peak power consumption.
